(19) United States
(12) Patent Application Publication
(10) Pub. No.: US 2025/0205222 A1
(43) Pub. Date: Jun. 26, 2025

(54) ANTIOBESITY COMPOSITIONS

(71) Applicant: Milton S. Jackson, Campbellsville, KY (US)

(72) Inventor: Milton S. Jackson, Campbellsville, KY (US)

(21) Appl. No.: 18/847,441

(22) PCT Filed: Mar. 7, 2023
(86) PCT No.: PCT/US23/14750

§ 371 Date: Sep. 16, 2024

Related U.S. Application Data
(60) Provisional application No. 63/321,533, filed on Mar. 18, 2022.

(51) Int. Cl. A61K 31/485; A61K 9/48; A61K 31/137; A61P 3/04

(52) U.S. Cl. CPC A61K 31/485; A61K 9/48; A61K 31/137; A61P 3/04

(57) ABSTRACT

Disclosed is a novel obesity medication with enhanced effectiveness, safety, and economics, and how to make and use the medication. The medication controls hunger, appetite, and cravings, promoting satiety, and meets with compliance by patients.

### CROSS REFERENCE TO RELATED APPLICATION(S)

This application claims priority to U.S. Provisional Application No. 63/321,533, filed Mar. 18, 2022, the contents of which are hereby incorporated by reference herein.

### BACKGROUND

Obesity is a medical problem resulting largely from overeating the wrong foods. Presently used obesity medicines are only weakly effective. Moreover, healthcare provider recommendations of such medicines have met with inadequate and/or inconsistent compliance (adherence to prescribed regimen) by patients. Currently there is a need for obesity medication and method of use thereof.

### SUMMARY

One aspect provides a dosage form composition for peroral administration to a subject in need of treatment for obesity, the dosage form composition comprising, consisting of, or consisting essentially of a therapeutically effective amount of naltrexone, a therapeutically effective amount of phentermine, and one or more pharmaceutically acceptable excipients.

One aspect provides a dosage form composition for peroral administration to a subject in need of treatment for obesity, the dosage form composition comprising, consisting of, or consisting essentially of a therapeutically effective amount of naltrexone hydrochloride, a therapeutically effective amount of phentermine hydrochloride, and one or more pharmaceutically acceptable excipients.

One aspect provides a method for inducing weight loss in a subject in need of weight loss, the method comprising administering to the subject a dosage form composition comprising, consisting of, or consisting essentially of a therapeutically effective amount of naltrexone hydrochloride, a therapeutically effective amount of phentermine hydrochloride, and one or more pharmaceutically acceptable excipients.

One aspect provides a method for inducing compliance with a healthcare provider recommendation of weight loss to a patient in need of weight loss, the method comprising the healthcare provider prescribing to the patient a dosage form composition comprising, consisting of, or consisting essentially of a therapeutically effective amount of naltrexone hydrochloride, a therapeutically effective amount of phentermine hydrochloride, and one or more pharmaceutically acceptable excipients; and the patient self-administering the dosage form composition for a period of time at the conclusion of which the patient has experienced weight loss, thereby complying with the healthcare provider recommendation.

### DESCRIPTION

To solve the problem of obesity, the present disclosure provides a novel obesity medication with enhanced effectiveness, safety, and economics. The medication controls hunger, appetite, and cravings, promoting satiety, and has met with compliance by patients.

In an embodiment, provided is a dosage form composition comprising a therapeutically effective amount of naltrexone and a therapeutically effective amount of phentermine and one or more pharmaceutically acceptable excipients.

One aspect provides a dosage form composition for peroral administration to a subject in need of treatment for obesity, the dosage form composition comprising a therapeutically effective amount of naltrexone hydrochloride, a therapeutically effective amount of phentermine hydrochloride, and one or more pharmaceutically acceptable excipients.

In certain embodiments, a therapeutically effective amount of naltrexone is from about 1.5 to about 4.5 mg, or any value there-between. In certain embodiments, the therapeutically effective amount of naltrexone is 1.5, 3.0, or 4.5 mg. In an embodiment, a therapeutically effective amount of phentermine is from about 15 to about 37.5 mg, or any value there-between. In certain embodiments, a therapeutically effective amount of phentermine is 15, 30, or 37.5 mg. In certain embodiments, the dosage form is in the form of a capsule.

One aspect provides a method for inducing weight loss in a subject in need of weight loss, the method comprising administering to the subject a dosage form composition comprising a therapeutically effective amount of naltrexone hydrochloride, a therapeutically effective amount of phentermine hydrochloride, and one or more pharmaceutically acceptable excipients.

In an embodiment, provided is a method for treating a subject in need of treatment for obesity, the method comprising administering to the subject a therapeutically effective amount of a composition comprising naltrexone, phentermine, and one or more pharmaceutically acceptable excipients.

In certain embodiments, administering occurs once per day. In an embodiment, administering occurs twice per day. In an embodiment, administering is peroral.

As used in this specification and the claims, “naltrexone” may refer to naltrexone base, having molecular formula C₂₀H₂₃NO₄ and CAS number 16590-41-3, or to naltrexone hydrochloride, having molecular formula C₂₀H₂₃NO₄×HCl and CAS number 16676-29-2, as dictated by context and by usage in the pharmaceutical and medical arts.

As used in this specification and the claims, “naltrexone hydrochloride” refers to naltrexone hydrochloride, having molecular formula C₂₀H₂₃NO₄×HCl and CAS number 16676-29-2.

As used in this specification and the claims, “phentermine” may refer to phentermine base, having molecular formula C₁₀H₁₅N and CAS number 122-09-8, or to phentermine hydrochloride, having molecular formula C₁₀H₁₅N·HCl and CAS number 1197-21-3, as dictated by context and by usage in the pharmaceutical and medical arts.

As used in this specification and the claims, “phentermine hydrochloride” refers to phentermine hydrochloride, having molecular formula C₁₀H₁₅N·HCl and CAS number 1197-21-3.

In an example, a composition comprising a therapeutically effective amount of naltrexone and a therapeutically effective amount of phentermine and one or more pharmaceutically acceptable excipients was made. Starting materials included the following per #100 size #1 capsules: Avicel PH105 (microcrystalline cellulose), sourced from Letco Medical, 23.976 g; phentermine hydrochloride USP, sourced from Medisca, 1.5 g; naltrexone hydrochloride USP, sourced from Letco Medical, 0.15 g; blue food coloring, sourced from Letco Medical, 0.024 g; size #1 capsules, sourced from Letco Medical. Mixing proceeded as follows: Avicel PH105 was first added to a mortar, followed by phentermine and naltrexone, and lastly blue food coloring to help confirm proper mixing; a pestle was used to reduce particle sizes and uniformly mix actives and excipients to make a powder. Capsule-filling proceeded as follows: ProFiller equipment was used; empty capsules were placed in ProFiller and separated; the powder was then dispersed into open capsules and pressed repeatedly using a pin tamper until no visible powder remained on the ProFiller platform; capsules were closed and secured using the machine. Packaging proceeded as follows: Each batch was given a lot number and stored in original plastic container from empty capsule wholesaler at controlled room temperature and humidity, with the appropriate Beyond Use Date of 180 days. Dispensing proceeded as follows: Capsules were dispensed as any other commercially-available capsules might be dispensed in the pharmacy setting—per prescription order and in plastic vials with proper labeling and childproof lids.

Compounding (or manufacture) as described in the paragraph immediately above may be modified to accommodate various masses and ratios of phentermine and naltrexone. Other pharmaceutically acceptable excipients may be substituted for those described in the paragraph immediately above, or, more generally, other combinations of excipients may be used in compounding or manufacture.

In an example, each of a series of patients was prescribed a filled capsule made as described above containing phentermine 15 mg/naltrexone 1.5 mg once daily for 7-10 days (for tolerance) and then twice daily. Typically, a patient self-administered this prescribed medication (the filled capsule) 1-2 hours before each of lunch and dinner. Another series of patients self-administered two such filled capsules together in the morning of each day. Another series of patients self-administered one such filled capsule per day, typically in the morning. Another series of patients self-administered one such filled capsule per day, before dinner. Another series of patients was prescribed a filled capsule made as described above but containing 37.5 mg phentermine and 3 mg naltrexone (per capsule) to be self-administered once daily. Meaningful weight loss was experienced by each series of patients. No serious adverse events were observed. Unexpectedly, each series of patients complied with its prescribed regimen, noting increased energy; a tendency to make more healthful dietary choices and to consume less food; and markedly decreased hunger, cravings and appetite.

Representative weight loss results experienced by a patient who was prescribed and who self-administered a medication (comprising naltrexone and phentermine) such as is disclosed herein are as follows, where “n” refers to the number of patients (it should be noted that this is a “snapshot” of patient weight loss after the given period of time, not a longitudinal study of the same patients over the course of time): After one month, 4.6% weight loss (n=14); after two months, 6.35% weight loss (n=17); after three months, 7.52% weight loss (n=10); after four months, 13.4% weight loss (n=1); after five months, 8.2% weight loss (n=2).

It will be recognized by one skilled in the art that a significant medical problem has been addressed by the subject of the present disclosure. Doses of naltrexone and phentermine were chosen to maximize effectiveness and minimize side-effects and “untoward” symptoms and therefore to maximize compliance and overall tolerance. Each of naltrexone and phentermine is subject to hepatic metabolism and renal excretion. Standard recommendation for duration of use may be 3 months, or up to 6 or 9 months if effective, but longer-term use may be indicated if continued effectiveness is demonstrated in addition to the observed favorable safety profile.

In accordance with the subject of the present disclosure it is contemplated that a patient may self-administer a total of from about 1.5 to about 4.5 mg naltrexone and from about 15 to about 37.5 mg phentermine per day. Such administration may be once a day, or such administration may be divided so as to be twice per day.

As used in this specification and the claims, the singular forms “a,” “an,” and “the” include plural referents unless the context clearly dictates otherwise. When used in this specification and the claims as an adverb rather than a preposition, “about” means “approximately” and comprises the stated value and every non-negative value within 10% of that value; in other words, “about 100%” includes 90% and 110% and every value in between. Unless stated otherwise, every range or interval includes both endpoints and every value in between.

As used herein the term “consisting essentially of” is defined to mean that specified materials may optionally be included in the composition that do not materially affect the basic and novel characteristics of the claims. Examples of such materials include preservatives and dispersants that do not have an impact on the function of the therapeutic composition.

Assays for active pharmaceutical ingredients such as are disclosed herein are described inter alia in the current edition of the United States Pharmacopeia, which is incorporated herein by reference.

A pharmaceutically acceptable excipient as is disclosed herein is described in the current edition of the Handbook of Pharmaceutical Excipients, published by, inter alia, the American Pharmacists Association, and which is incorporated herein by reference.

Subsequent to compounding or manufacture, a dosage form composition such as is disclosed herein may be placed in a suitable container-closure system such as is known in the art. Processes for filling such a container-closure system are known in the art.

Many modifications and other embodiments of what is disclosed herein will come to mind to one skilled in the art to which this disclosure pertains having the benefit of the teachings presented in the foregoing description. Therefore, it is to be understood that the scope of what has been conceived and reduced to practice is not to be limited to the specific embodiments disclosed herein and that modifications and other embodiments are intended to be included within the scope of the appended claims. Although specific terms are employed herein, they are used in a generic and descriptive sense only and not for purposes of limitation.

What is claimed is:

1. A dosage form composition for peroral administration to a subject in need of treatment for obesity, the dosage form composition comprising a therapeutically effective amount of naltrexone, a therapeutically effective amount of phentermine, and one or more pharmaceutically acceptable excipients.

2. The dosage form composition of claim 1, wherein the therapeutically effective amount of naltrexone is from about 1.5 to about 4.5 mg. 3 The dosage form composition of claim 2, wherein the therapeutically effective amount of naltrexone is 1.5, 3.0, or 4.5 mg.

4. The dosage form composition of claim 1, wherein the therapeutically effective amount of phentermine is from about 15 to about 37.5 mg.

5. The dosage form composition of claim 4, wherein the therapeutically effective amount of phentermine is 15, 30, or 37.5 mg.

6. The dosage form composition of claim 1 in the form of a capsule.

7. A dosage form composition for peroral administration to a subject in need of treatment for obesity, the dosage form composition comprising a therapeutically effective amount of naltrexone hydrochloride, a therapeutically effective amount of phentermine hydrochloride, and one or more pharmaceutically acceptable excipients.

8. The dosage form composition of claim 7, wherein the therapeutically effective amount of naltrexone hydrochloride is from about 1.5 to about 4.5 mg.

9. The dosage form composition of claim 8, wherein the therapeutically effective amount of naltrexone hydrochloride is 1.5, 3.0, or 4.5 mg.

10. The dosage form composition of claim 7, wherein the therapeutically effective amount of phentermine hydrochloride is from about 15 to about 37.5 mg.

11. The dosage form composition of claim 10, wherein the therapeutically effective amount of phentermine hydrochloride is 15, 30, or 37.5 mg.

12. The dosage form composition of claim 7 in the form of a capsule.

13. A method for inducing weight loss in a subject in need of weight loss, the method comprising administering to the subject a dosage form composition comprising a therapeutically effective amount of naltrexone hydrochloride, a therapeutically effective amount of phentermine hydrochloride, and one or more pharmaceutically acceptable excipients.

14. The method of claim 13, wherein the administering occurs once per day.

15. The method of claim 13, wherein the administering occurs twice per day.

16. The method of claim 13, wherein the therapeutically effective amount of naltrexone hydrochloride is from about 1.5 to about 4.5 mg.

17. The method of claim 16, wherein the therapeutically effective amount of naltrexone hydrochloride is 1.5, 3.0, or 4.5 mg.

18. The method of claim 13, wherein the therapeutically effective amount of phentermine hydrochloride is from about 15 to about 37.5 mg

19. The method of claim 18, wherein the therapeutically effective amount of phentermine hydrochloride is 15, 30, or 37.5 mg.

20. The method of claim 13, wherein the dosage form composition is in the form of a capsule.

21. A method for inducing compliance with a healthcare provider recommendation of weight loss to a patient in need of weight loss, the method comprising the healthcare provider prescribing to the patient a dosage form composition comprising a therapeutically effective amount of naltrexone hydrochloride, a therapeutically effective amount of phentermine hydrochloride, and one or more pharmaceutically acceptable excipients; and
the patient self-administering the dosage form composition for a period of time at the conclusion of which the patient has experienced weight loss, thereby complying with the healthcare provider recommendation.

22. The method of claim 21, wherein the self-administering occurs once per day.

23. The method of claim 21, wherein the self-administering occurs twice per day.

24. The method of claim 21, wherein the therapeutically effective amount of naltrexone hydrochloride is from about 1.5 to about 4.5 mg.

25. The method of claim 24, wherein the therapeutically effective amount of naltrexone hydrochloride is 1.5, 3.0, or 4.5 mg.

26. The method of claim 21, wherein the therapeutically effective amount of phentermine hydrochloride is from about 15 to about 37.5 mg.

27. The method of claim 26, wherein the therapeutically effective amount of phentermine hydrochloride is 15, 30, or 37.5 mg.

28. The method of claim 21, wherein the dosage form composition is in the form of a capsule.
